(19) United States
(12) Patent Application Publication
(10) Pub. No.: US 2010/0266620 A1
(43) Pub. Date: Oct. 21, 2010

(54) Immunoconjugates for treatment of infectious diseases

(76) Inventor: Le Sun, South Plainfield, NJ (US)

Correspondence Address: WINSTON & STRAWN LLP;PATENT DEPARTMENT, 1700 K STREET, N.W., WASHINGTON, DC, 20006, US

(21) Appl. No.: 11/655,266

(22) Filed: Jan. 19, 2007

Related U.S. Application Data
(60) Provisional application No. 60/760,383, filed on Jan. 20, 2006.

(51) Int. Cl. A61K 39/395; C07K 16/08; C07K 16/10; A61P 31/12

(52) U.S. Cl. 424/178.1; 530/391.7

(57) ABSTRACT

The present invention features immunoconjugates which comprise therapeutic agents coupled to antibodies that specifically recognize pathogen surface antigens. These immunoconjugates can be used to treat or prevent infectious diseases. Upon administration of an immunoconjugate of the present invention to an infected host, the immunoconjugate binds to a specific antigen on the surface of the targeted pathogen (e.g., virus). As the pathogen enters the host cells, the therapeutic agent(s) in the immunoconjugate destroys the infected host cells (and, preferably, the bound pathogen), thereby preventing the replication and transmission of the pathogen. In one embodiment, the immunoconjugates of the present invention specifically recognize surface/envelope antigens of the following viruses: HIV, HBV, HCV, EBV, influenza virus, and SARS associated coronavirus.

### CROSS REFERENCE TO RELATED APPLICATIONS

The present application claims priority from and incorporates by reference the entire disclosure of U.S. Provisional Patent Application Ser. No. 60/760,383, filed Jan. 20, 2006.

### TECHNICAL FIELD

The present invention relates to immunoconjugates comprising therapeutic agents coupled to antibodies, and methods of using these immunoconjugates to treat and prevent infectious diseases.

### BACKGROUND OF THE INVENTION

Human immunodeficiency virus (HIV) is a retrovirus and primarily infects the human immune system such as CD4⁺T cells, macrophages and dendritic cells. HIV infection is characterized as a systemic immunosuppressive disorder caused by the viral-mediated depletion of CD4 T cells or viral mediated loss of immune competence, which develops into the profound immunodeficiency that underlies the acquired immunodeficiency syndrome (AIDS). AIDS is characterized by various pathological conditions, including immune incompetence, opportunistic infections, neurological dysfunctions, and neoplastic growth.

Hepatitis B virus (HBV) is a member of the Hepadnavirus family. HBV is largely transmitted through exposure to bodily fluids containing the virus. HBV infection may either be acute or chronic. Acute infection with HBV is associated with acute viral hepatitis. Chronic infection with HBV can be either asymptomatic or associated with chronic hepatitis, leading to cirrhosis over a period of several years. Chronic infection may dramatically increase the incidence of liver cancer.

Hepatitis C virus (HCV) is a major cause of chronic liver disease worldwide. Approximately 170 million individuals are infected worldwide by HCV. HCV infection causes acute hepatitis, which has a high probability of becoming chronic and in the long term can lead to cirrhosis and hepatocellular carcinoma.

Epstein-Barr virus (EBV) is a virus of the herpes family, and is one of the most common viruses in humans. EBV primarily infects B cells, and can cause infectious mononucleosis. EBV is also thought to be associated with the formation of a variety of tumors, such as lymphomas, leukemias, carcinomas, and sarcomas. In addition, EBV has been implicated as a co-factor in invasive breast cancer.

Much effort has been made to treat and prevent HIV, HBV, HCV, and EBV infections. However, all the effort has showed limited success. Therefore, significant challenges still remain in the scientific and clinical battle against these viral diseases.

### SUMMARY OF THE INVENTION

The present invention features immunoconjugates which comprise therapeutic agents coupled to antibodies that specifically recognize pathogen surface antigens. These immunoconjugates can be used to treat and prevent infectious diseases. Upon administration of an immunoconjugate of the present invention to an infected host (e.g., a human patient), the immunoconjugate binds to a specific antigen on the surface of the targeted pathogen (e.g., a virus). The therapeutic agent(s) comprised in the immunoconjugate may destroy the bound pathogen as well as the infected host cells as the pathogen enters the host cells, thereby effectively preventing the replication and further transmission of the pathogen.

As the pathogen enters the host cells, the therapeutic agent(s) comprised in the immunoconjugate destroys the infected host cells and, preferably, the bound pathogen, thereby effectively preventing the replication and further transmission of the pathogen.

In one aspect, the immunoconjugates of the present invention comprise an antibody coupled to a therapeutic agent, where the antibody specifically recognizes a surface or envelope antigen of a virus. The therapeutic agent can be, without limitation, a radioactive isotope, a cytotoxic agent, or a prodrug. Suitable radioactive isotopes include, but are not limited to, At²¹¹, I¹³¹, I¹²⁵, Y⁹⁰, Re¹⁸⁶, Re¹⁸⁸, Sm¹⁵³, Bi²¹², P³², and Lu¹⁷⁷. Non-limiting examples of suitable cytotoxic agent include mitomycin C and pingyangmycin.

In one embodiment, an immunoconjugate of the present invention comprises an antibody that specifically recognizes a surface/envelope antigen selected from the group consisting of:

an HIV surface or envelope antigen;

an HBV surface or envelope antigen;

an HCV surface or envelope antigen;

an EBV surface or envelope antigen;

an influenza surface or envelope antigen; and

a SARS associated coronavirus surface or envelope antigen.

In another embodiment, an immunoconjugate of the present invention comprises an antibody that specifically recognizes a viral surface antigen selected from the group consisting of HIV gp120, HIV gp41, HBV surface protein antigen, HCV E1, HCV E2, EBV gp350, EBV gp220, EBV gp85, EBV gp25, EBV gp42, influenza hemagglutinin, and influenza neuraminidase, coronavirus spike glycoprotein, and coronavirus hemagglutinin-acetylesterase.

In still another embodiment, an immunoconjugate of the present invention comprises an antibody selected from the group consisting of anti-HIV mAb 2F5, anti-HIV mAb 2G12, anti-HIV mAb 4E10, anti-HIV mAb C108G, and anti-HBVsAg mAb.

Antibodies suitable for the present invention include, but are not limited to, monoclonal antibodies (mAb), polyclonal antibodies, multispecific antibodies (e.g. bispecific antibodies), chimeric antibodies, linear antibodies, reduced antibodies (e.g., rIgG), Fab fragments, Fab′ fragments, F(ab′)₂, Fv fragments, single-chain Fv (scFv) molecules, diabodies, triabodies, minibodies, and other antibody fragments that comprise antigen-binding sites. In one embodiment, the antibody employed in the present invention is selected from the group consisting of IgG, IgM, IgA, IgD, and IgE. In another embodiment, the antibody employed in the present invention is selected from the group consisting of full-length antibody, scFv, Fv, Fab, F(ab)₂, Fab′, diabody, triabody, and minibody. In yet another embodiment, the antibody employed in the present invention is a rat, murine, cow, dog, sheep, goat, guinea pig, rabbit, macaque, chimpanzee, or chimeric antibody. In a preferred embodiment, the antibody employed in the present invention is a humanized or human antibody.

The present invention also features pharmaceutical compositions comprising the immunoconjugates of the present invention.

In addition, the present invention features methods for treating or preventing viral infections. In one embodiment, the methods comprise contacting a virus with an immunoconjugate of the present invention, where the immunoconjugate comprises an antibody specifically recognizing a surface or envelope antigen of the virus. In another embodiment, the methods comprise administering an immunoconjugate of the present invention to a subject of interest, where the immunoconjugate comprises an antibody specifically recognizing a surface or envelope antigen of the targeted virus. The subject being treated can be a human or an animal.

Other features, objects, and advantages of the present invention are apparent in the detailed description that follows. It should be understood, however, that the detailed description, while indicating embodiments of the present invention, is given by way of illustration only, not limitation. Various changes and modifications within the scope of the invention will become apparent to those skilled in the art from the detailed description.

### DETAILED DESCRIPTION OF THE INVENTION

The present invention features immunoconjugates comprising antibodies coupled to therapeutic agents, where the antibodies can specifically bind to surface or envelope antigens of viruses or other infectious pathogens. The present invention also features methods of using these immunoconjugates to treat or prevent infectious diseases. An immunoconjugate of the present invention can be administered to an infected host to allow the immunoconjugate to specifically bind to the targeted pathogen. As the pathogen enters the host cells, the therapeutic agent(s) in the immunoconjugate kills or neutralizes the host cells, thereby preventing the replication and further transmission of the pathogen. In many embodiments, the therapeutic agent(s) also destroys or irreversibly damages the bound pathogen.

Antibodies suitable for the present invention include, but are not limited to, monoclonal antibodies (mAb), polyclonal antibodies, multispecific antibodies (e.g. bispecific antibodies), chimeric antibodies, linear antibodies, reduced antibodies (e.g., rIgG), Fab fragments, Fab′ fragments, F(ab′)₂, Fv fragments; single-chain Fv (scFv) molecules, diabodies, triabodies, minibodies, and other antibody fragments that comprise antigen-binding sites. The present invention features the use of any antibody isotype, e.g., IgG, IgM, IgA, IgD, or IgE. Preferably, an antibody of the present invention (including Fab, Fv, or other antigen-binding fragments) binds to the target antigen with an affinity of at least 10⁻⁵ M⁻¹, 10⁻⁶ M⁻¹, 10⁻⁷ M⁻¹, 10⁻⁸ M⁻¹, 10⁻⁹ M⁻¹, 10⁻¹⁹ M⁻¹, or stronger. An antibody of the present invention can be a rat, murine, cow, dog, sheep, goat, guinea pig, rabbit, macaque, chimpanzee, chicken, or human antibody. Antibodies derived from other non-human primates, mammals, or vertebrates are also contemplated by the present invention. Methods for making intact antibodies or antigen-binding fragments are well known in the art.

The basic unit of a native antibody is a monomer which consists of two identical heavy chains and two identical light chains linked by disulfide bonds. There are at least five different types of heavy chains—namely, γ, α, δ, μ, and ε, which provide different effector functions. Heavy chains γ, α and δ have three constant domains (C_{H}1, C_{H}2, and C_{H}3), and heavy chains g and c have four constant domains (C_{H}1, C_{H}2, C_{H}3, and C_{H}4). Each heavy chain also has one variable domain (V_{H}). There are at least two types of light chain—namely, λ and κ, each of which comprises one constant domain (C_{L}) and one variable domain (V_{L}).

Depending on the amino acid sequences of the constant domains of their heavy chains, native mammalian antibodies can be grouped into five classes: IgG, IgA, IgM, IgD, and IgE. Several of these classes can be further divided into subclasses or isotypes, such as IgG1, IgG2, IgG3, IgG4, IgA, or IgA2. A typical IgG molecule is composed of two heavy chains γ and two identical light chains (λ or κ). Disulfide bonds connect the light chains to the heavy chains, as well as between the heavy chains. The constant domain of the light chain is aligned with the first constant domain of the heavy chain, and the light-chain variable domain (V_{L}) is aligned with the variable domain of the heavy chain (V_{H}) to form the antigen recognition site.

The variability in a variable domain (V_{L} or V_{H}) is not evenly distributed throughout the domain. It is typically concentrated in three segments called hypervariable regions. The more highly conserved portions of a variable domain are called the framework regions (FRs). Each variable domain of native heavy and light chains comprises four FRs, largely adopting a β-sheet configuration, connected by three hypervariable regions, which form loops connecting, and in some cases forming part of, the β-sheet structure. The hypervariable regions in each chain are held together in close proximity by the FRs and, with the hypervariable regions from the other chain, contribute to the formation of the antigen-binding site of the antibody. See Kabat et al., SEQUENCES OF PROTEINS OF IMMUNOLOGICAL INTEREST, 5th Ed. Public Health Service, National Institutes of Health, Bethesda, Md. (1991).

Papain digestion of an antibody produces two identical antigen-binding fragments, called Fab fragments, each with a single antigen-binding site, and a residual Fc fragment. Pepsin treatment of an antibody yields an F(ab′)₂ fragment which has two antigen-binding sites joined at the hinge region between C_{H}1 and C_{H}2 through disulfide bonds. The reduction of the F(ab′)₂ fragment produces two Fab′ fragments. Each Fab′ fragment contains at least one sulfhydryl group that can be utilized in conjugation with a toxin, a radioactive isotope, or another agent of interest. Fab′ fragments differ from Fab fragments by the addition of a few residues at the carboxyl terminus of the heavy chain C_{H}1 domain. Fab, Fab′, F(ab′)₂, and other antigen-binding fragments can also be produced using recombinant host cells, as appreciated by those skilled in the art.

Methods for making intact antibodies or antigen-binding fragments are well known in the art. As used herein, “monoclonal” should not be construed as requiring production of the antibody by any particular method. Exemplary methods suitable for making monoclonal antibodies include, but are not limited to, the hybridoma methods (e.g., Kohler et al., NATURE, 256:495 (1975)), the recombinant DNA methods (see, e.g., U.S. Pat. No. 4,816,567), and the phage antibody library methods (e.g., Clackson et al., NATURE, 352:624-628 (1991), and Marks et al., J. MOL. BIOL., 222:581-597 (1991)).

DNA encoding the monoclonal antibodies can be readily isolated and sequenced using conventional procedures (e.g., by using oligonucleotide probes that are capable of binding specifically to genes encoding the heavy and light chains of murine antibodies). The hybridoma cells used for the production of the monoclonal antibodies serve as a preferred source of such DNA. Once isolated, the DNA may be placed into expression vectors, which are then transfected into host cells such as E. coli cells, simian COS cells, Chinese Hamster Ovary cells, or myeloma cells that do not otherwise produce antibody protein, to obtain the synthesis of monoclonal antibodies in the recombinant host cells. The DNA thus isolated can also be used to prepare chimeric antibodies, antigen-binding fragments, or other antibody derivatives, as appreciated by those skilled in the art.

A chimeric antibody refers to an antibody in which a portion of the heavy or light chain is identical or homologous to corresponding sequences in antibodies derived from a particular species or belonging to a particular antibody class or subclass, while the remainder of the chain(s) is identical or homologous to corresponding sequences in antibodies derived from another species or belonging to another antibody class or subclass. Non-limited examples of chimeric antibodies include those described in U.S. Pat. No. 4,816,567 and Morrison et al., PROC. NATL. ACAD. SCI. USA, 81:6851-6855 (1984).

Preferably, a chimeric antibody of the present invention is a humanized antibody. Humanized antibodies are particularly desirable for therapeutic treatment of human subjects. Humanized forms of non-human (e.g., murine) antibodies are chimeric full-length immunoglobulins, or chimeric antigen-binding fragments (such as Fv, Fab, or F(ab′)₂), which contain minimal sequence derived from non-human immunoglobulin. Humanized antibodies can be derived from human immunoglobulins in which the residues forming the complementary determining regions (CDRs) are replaced by the residues from CDRs of a non-human antibody. In some instances, Fv framework residues of the human immunoglobulin are also replaced by corresponding non-human residues. Humanized antibodies may include residues that are found neither in the recipient antibody nor in the imported CDR or framework sequences. A humanized antibody can comprise at least one or two variable domains, in which all or substantially all of the CDR regions correspond to those of a non-human immunoglobulin and all or substantially all of the constant regions are those of a human immunoglobulin consensus sequence. In many embodiments, a humanized antibody of the present invention comprises at least a portion of a constant region of a human immunoglobulin (Fc). For further details, see Jones et al., NATURE 321:522-525 (1986); Riechmann et al., NATURE 332:323-329 (1988); and Presta, CURR. OP. STRUCT. BIOL. 2:593-596 (1992).

The present invention also features the use of human antibodies. Human antibodies can be produced using transgenic mice, which are incapable of expressing endogenous immunoglobulin heavy and light chains but can express human heavy and light chains. The transgenic mice are immunized in the normal fashion with a selected antigen. Monoclonal antibodies directed against the antigen can be obtained using conventional hybridoma technology. The human immunoglobulin transgenes harbored in the transgenic mice rearrange during B cell differentiation, and subsequently undergo class switching and somatic mutation. Using this technique, therapeutically useful IgG, IgA, IgE, or other antibody isotypes can be prepared. Alternatively, phage display technology (McCafferty et al., NATURE 348:552-553 (1990)) can be used to produce intact human antibodies and antibody fragments in vitro, from immunoglobulin variable (V) domain gene repertoires from unimmunized donors. Phage display can be performed in a variety of formats. See, e.g., Johnson, Kevin S, and Chiswell, David J., CURRENT OPINION IN STRUCTURAL BIOLOGY, 3:564-571 (1993). Several sources of V-gene segments can be used for phage display. A repertoire of V genes from unimmunized human donors can be constructed and antibodies to a diverse array of antigens (including self-antigens) can be isolated essentially following the techniques described by Marks et al., J. MOL. BIOL., 222:581-597 (1991), or Griffith et al., EMBO J. 12:725-734 (1993). In addition, human antibodies can be generated by in vitro activated B cells (see U.S. Pat. Nos. 5,567,610 and 5,229,275).

Multispecific (e.g., bispecific) antibodies are antibodies that have binding specificities for at least two different epitopes. Traditional production of full length bispecific antibodies is based on the coexpression of two immunoglobulin heavy chain-light chain pairs, where the two chains have different specificities. Because of the random assortment of immunoglobulin heavy and light chains, these hybridomas (quadromas) produce a potential mixture of 10 different antibody molecules, of which only one has the correct bispecific structure. Purification of the correct molecule, which is usually done by affinity chromatography steps, is rather cumbersome. According to a different approach, antibody variable domains with the desired binding specificities (antibody-antigen combining sites) are fused to immunoglobulin constant domain sequences. The fusion preferably is with an immunoglobulin heavy chain constant domain, comprising at least part of the hinge, CH2, and CH3 regions. It is preferred to have the first heavy-chain constant region (CH1) containing the site necessary for light chain binding, present in at least one of the fusions. DNAs encoding the immunoglobulin heavy chain fusions and, if desired, the immunoglobulin light chain, are inserted into separate expression vectors, and are co-transfected into a suitable host organism. This provides for great flexibility in adjusting the mutual proportions of the three polypeptide fragments in embodiments when unequal ratios of the three polypeptide chains used in the construction provide the optimum yields. In one embodiment, the bispecific antibodies are composed of a hybrid immunoglobulin heavy chain with a first binding specificity in one arm, and a hybrid immunoglobulin heavy chain-light chain pair (providing a second binding specificity) in the other arm. This asymmetric structure can facilitate the separation of the desired bispecific compound from unwanted immunoglobulin chain combinations, as the presence of an immunoglobulin light chain in only one half of the bispecific molecule provides for a facile way of separation.

Multispecific (e.g., bispecific) antibodies also encompass cross-linked or “heteroconjugate” antibodies. For example, one of the antibodies in the heteroconjugate can be coupled to avidin, the other to biotin. Heteroconjugate antibodies may be made using any convenient cross-linking methods. Suitable cross-linking agents are well known in the art, and are disclosed in U.S. Pat. No. 4,676,980, along with a number of cross-linking techniques. Multispecific antibodies can also be produced from recombinant cell culture. See, e.g., Kostelny et al., J. IMMUNOL., 148(5):1547-1553 (1992).

Reduced antibodies are a reduced form of immunoglobulin composed of one complete light chain and one complete heavy chain connected by disulfide bonds. It is essentially one-half of an intact immunoglobulin molecule and contains a single antigen-binding site. Reduced antibodies can be formed by the selective reduction of disulfide bonds in the hinge region of an antibody.

An Fv fragment contains a complete antigen-binding site which includes a V_{L} domain and a V_{H} domain held together by non-covalent interactions. The present invention also features Fv fragments in which the V_{L} and V_{H} domains are cross linked through glutaraldehyde, intermolecular disulfides, or other linkers.

The variable domains of the heavy and light chains can be fused together to form a single chain variable fragment (scFv), which retains the original specificity of the parent immunoglobulin. Preferably, the V_{L} domain is connected to the V_{H} domain by a flexible peptide linker of 5-30 amino acids in length. More preferably, the V_{L} domain is connected to the V_{H} domain by a flexible peptide linker of 10-20 amino acids in length. Highly preferably, the V_{L} domain is connected to the V_{H} domain by a flexible peptide linker of about 15 amino acids in length. Linkers with less than 5, or more than 30, amino acid residues may also be used, provided that they enables the scFv to form the desired structure for antigen binding. For a review of scFv see Plückthun in THE PHARMACOLOGY OF MONOCLONAL ANTIBODIES, vol. 113, Rosenburg and Moore eds., Springer-Verlag, New York, pp. 269-315 (1994).

Diabodies are small antibody fragments with two antigen-binding sites, where each fragment comprises a variable heavy domain (V_{H}) connected to a variable light domain (V_{L}) in the same polypeptide chain (V_{H}-V_{L}). By using a linker that is too short to allow pairing between the two domains on the same chain, the domains are forced to pair with the complementary domains of another chain and create two antigen-binding sites. Triabodies can be similarly constructed with three antigen-binding sites.

The basic unit of a preferred minibody comprises a V_{L} and a V_{H} domain. In many cases, the basis unit of a minibody also comprises one or more C_{H} or C_{L} domain. For instance, the basic unit of a minibody can be V_{L}-V_{H}-C_{H1}, V_{L}-V_{H}-C_{H2}, or V_{L}-V_{H}-C_{H3}. Each minibody may include 1, 2 or more such units to form 1, 2, or more antigen-binding sites.

Therapeutic agents suitable for the present invention include, but are not limed to, radioactive isotopes, cytotoxic agents, or prodrugs. Non-limiting examples of suitable radioactive isotopes include At²¹¹, I¹³¹, I¹²⁵, Y⁹⁰, Re¹⁸⁶, Re¹⁸⁸, Sm¹⁵³, Bi²¹², P³², and Lu¹⁷⁷. Non-limiting examples of suitable cytotoxic agents include chemotherapeutic agents, toxins, or other substances that can inhibit or prevent the function of cells or causes destruction of cells. Specific examples of chemotherapeutic agents include, but are not limited to, alkylating agents such as thiotepa and cyclosphosphamide (CYTOXAN™); alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphaoramide and trimethylolomelamine; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as aclacinomysins, actinomycin, authramycin, azaserine, bleomycins, cactinomycin, calicheamicin, carabicin, carnomycin, carzinophilin, chromomycins, dactinomycin, daunorubicin, detorubicin, 6-diazo-5-oxo-L-norleucine, doxorubicin, epirubicin, esorubicin, idarubicin, marcellomycin, mitomycins, mycophenolic acid, nogalamycin, olivomycins, peplomycin, pingyangmycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine, 5-FU; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; amsacrine; bestrabucil; bisantrene; edatraxate; defofamine; demecolcine; diaziquone; elformithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; PSK®; razoxane; sizofuran; spirogermanium; tenuazonic acid; triaziquone; 2,2′,2″-trichlorotriethylamine; urethan; vindesine; dacarbazine; mannomustine; mitobronitol; mitolactol; pipobroman; gacytosine; arabinoside (“Ara-C”); cyclophosphamide; thiotepa; taxanes, e.g. paclitaxel (TAXOL®, Bristol-Myers Squibb Oncology, Princeton, N.J.) and docetaxel (TAXOTERE®, Sanofi-aventis); chlorambucil; gemcitabine; 6-thioguanine; mercaptopurine; methotrexate; platinum analogs such as cisplatin and carboplatin; vinblastine; platinum; etoposide (VP-16); ifosfamide; mitomycin C; mitoxantrone; vincristine; vinorelbine; navelbine; novantrone; teniposide; daunomycin; aminopterin; xeloda; ibandronate; CPT-11; topoisomerase inhibitor RFS 2000; difluoromethylornithine (DMFO); retinoic acid; esperamicins; capecitabine; hormones (such as steroids); anthracycline; vinca alkaloids; mithramycin; neocarzinostatin; macromycin; trenimon; α-amanitin; ricin; ricin A-chain; ethiduim bromide; tenoposide; colchicine; dihydroxy anthracin dione; actinomycin D; diphteria toxin; abrin; arbrin A chain; modeccin A chain; alpha-sarcin; gelonin; mitogellin; retstrictocin; phenomycin; enomycin; α-sarcin; aspergillin; restirictocin; ribonuclease; diphtheria toxin; pseudomonas exotoxin; curicin; crotin; calicheamicin; sapaonaria officinalis inhibitor; maytansinoids; goserelin; glucocorticoids; and pharmaceutically acceptable salts, acids or derivatives of any of the above. Toxins suitable for the present invention include small molecule toxins or enzymatically active toxins of bacterial, fungal, plant or animal origin, including fragments and/or variants thereof. Specific examples of preferred toxins include, but are not limited to, mitomycin C, pingyangmycin, calicheamicin, maytansine (U.S. Pat. No. 5,208,020), trichothene, and CC 1065.

The therapeutic agents employed in the present invention can also be prodrugs. A prodrug is a precursor or derivative form of a pharmaceutically active substance that is less cytotoxic than the parent drug and is capable of being enzymatically activated or converted into the more active parent form. See, e.g., U.S. Pat. No. 4,975,287, which is incorporated herein by reference in its entirety. Non-limiting examples of prodrugs include phosphate-containing prodrugs, thiophosphate-containing prodrugs, sulfate-containing prodrugs, peptide-containing prodrugs, D-amino acid-modified prodrugs, glycosylated prodrugs, β-lactam-containing prodrugs, optionally substituted phenoxyacetamide-containing prodrugs or optionally substituted phenylacetamide-containing prodrugs, 5-fluorocytosine and other 5-fluorouridine prodrugs which can be converted into the more active cytotoxic free drug. Examples of cytotoxic drugs that can be derivatized into a prodrug form for use in this invention include, but are not limited to, those chemotherapeutic agents described above.

In addition, the therapeutic agents employed in the present invention can be prodrug-activating enzymes which convert prodrugs (e.g. a peptidyl chemotherapeutic agent) to active drugs. Enzymes that are suitable for this purpose include, but are not limited to, alkaline phosphatase useful for converting phosphate-containing prodrugs into free drugs; arylsulfatase useful for converting sulfate-containing prodrugs into free drugs; cytosine deaminase useful for converting non-toxic 5-fluorocytosine into the anti-cellular drug, 5-fluorouracil; proteases, such as serratia protease, thermolysin, subtilisin, carboxypeptidases and cathepsins (such as cathepsins B and L), that are useful for converting peptide-containing prodrugs into free drugs; D-alanylcarboxypeptidases, useful for converting prodrugs that contain D-amino acid substituents; carbohydrate-cleaving enzymes such as β-galactosidase and neuraminidase useful for converting glycosylated prodrugs into free drugs, β-lactamase useful for converting drugs derivatized with β-lactams into free drugs; and penicillin amidases, such as penicillin Vamidase or penicillin G amidase, useful for converting drugs derivatized at their amine nitrogens with phenoxyacetyl or phenylacetyl groups, respectively, into free drugs.

An antibody employed in the present invention can be conjugated with one type of therapeutic agents (e.g., radioactive isotopes, chemotherapeutic agents, toxins, or prodrugs). An antibody employed in the present invention can also be conjugated with two or more different types of therapeutic agents (e.g., radioactive isotopes+chemotherapeutic agents, radioactive isotopes+toxin, radioactive isotopes+prodrugs, chemotherapeutic agents/toxins+prodrugs, or radioactive isotopes+chemotherapeutic agents/toxins+prodrugs). In addition, two or more different agents of the same type (e.g., two different radioisotopes) can be coupled to an antibody employed in the present invention.

Conjugation of therapeutic agents to an antibody can be achieved by a variety of mechanisms, for example, covalent coupling, affinity binding, intercalation, coordinate binding, or complexation. Preferred conjugation methods are those involving covalent coupling, such as using chemical cross-linkers, natural peptides, or disulfide bonds.

Covalent coupling can be achieved either by direct condensation of existing side chains or by the incorporation of external bridging molecules. Many bivalent or polyvalent agents are useful in coupling protein molecules to other proteins, peptides or amine functions.

Examples of coupling agents are carbodiimides, diisocyanates, glutaraldehyde, diazobenzenes, and hexamethylene diamines. Other suitable cross-linking agents include SPDP (N-succinimidyl-3-(2-pyridyldithiol)propionate), EDC (1-Ethyl-3-[(3-dimethylaminopropyl]carbodiimide Hydrochloride), AEDP (3-[(2-Aminoethyl)dithio]propionic acid.HCl), ASBA (4-[p-Azidosalicylamido]butylamine), DCC, BMPS(N-[β-Maleimidopropyloxy]succinimide ester), EMCS ([N-e-Maleimidocaproyloxy]succinimide ester), LC-SMCC (Succinimidyl-4-[N-Maleimidomethyl]cyclohexane-1-carboxy-[6-amidocaproate]), LC-SPDP (Succinimidyl 6-(3-[2-pyridyldithio]-propionamido)hexanoate), MBS (m-Maleimidobenzoyl-N-hydroxysuccinimide ester), SIAB (N-Succinimidyl[4-iodoacetyl]aminobenzoate), SMCC (Succinimidyl 4-[N-maleimidomethyl]cyclohexane-1-carboxylate), SMPH (Succinimidyl-6-[β-maleimidopropionamido]hexanoate), Sulfo-SMCC (Sulfosuccinimidyl 4-[N-maleimidomethyl]cyclohexane-1-carboxylate), Sulfo-SMPB (Sulfosuccinimidyl 4-[p-maleimidophenyl]butyrate), Sulfo-NHS-LC-ASA (Sulfosuccinimidyl[4-azidosalicylamido]-hexanoate), Sulfo-SASD (Sulfosuccinimidyl-2-[p-azidosalicylamido]ethyl-1,3′-dithiopropionate), PMPI (N-[p-Maleimidophenyl]isocyanate), NHS-ASA (N-Hydroxysuccinimidyl-4-azidosalicylic acid), SMPT, Sulfo-LC-SPDP, Sulfo-MBS, Sulfo-SIAB, EDC/Sulfo-NHS, NHS-PEOₙ-Maleimide (n=2, 4, 8, or 12), APG (p-Azidophenyl glyoxal monohydrate), ABH (p-Azidobenzoyl hydrazide), TFCS(N-[e-Trifluoroacetylcaproyloxy]succinimide ester), MPBH 4-(4-N-Maleimidophenyl)butyric acid hydrazide hydrochloride, PDPH (3-(2-Pyridyldithio)propionyl hydrazide), and EMCH ([N-e-Maleimidocaproic acid]hydrazide). This list is not intended to be exhaustive of the various coupling agents known in the art but, rather, is exemplary of the more common coupling agents that may be used.

In many embodiments, hetero-bifunctional cross-linkers are employed, which contain two reactive groups: one generally reacting with primary amine group (e.g., N-hydroxy succinimide) and the other generally reacting with a thiol group (e.g., pyridyl disulfide, maleimides, or halogens.). Through the primary amine reactive group, the cross-linker can react with the lysine residue(s) of the antibody and through the thiol reactive group, the cross-linker, already tied up to the antibody, reacts with the cysteine residue (or other free sulfhydryl group) on a therapeutic agent (e.g. a cytotoxic or anti-cellular agent).

In many embodiments, an antibody is first derivatized, following by attachment of the therapeutic agent(s) to the derivatized product. As used herein, the term “derivatize” refers to the chemical modification of the antibody substrate with a suitable cross-linking agent. Examples of cross-linking agents for use in this manner include the disulfide-bond containing linkers SPDP (N-succinimidyl-3-(2-pyridyldithio)propionate) and SMPT (4-succinimidyl-oxycarbonyl-α-methyl-α(2-pyridyldithio)toluene). Lysinyl (or amino-terminal) residues are reactive with succinic or other carboxylic acid anhydrides. Derivatization with these agents has the effect of reversing the charge of the lysinyl residues. Other suitable reagents for derivatizing amino-containing residues include imidoesters such as methyl picolinimidate, pyridoxal phosphate, pyridoxal, chloroborohydride, trinitrobenzenesulfonic acid, O-methylisourea, 2,4-pentanedione, and transaminase-catalyzed reaction with glyoxylate. Modification of tyrosyl residues can be made by reaction with aromatic diazonium compounds or tetranitromethane. For instance, N-acetylimidizole and tetranitromethane can be used to form O-acetyl tyrosyl species and 3-nitro derivatives, respectively. Tyrosyl residues can also be iodinated using I¹²⁵ or I¹³¹.

In addition, a sequence enriched with lysine or tyrosine residues can be added to an antibody for additional coupling of therapeutic agents. This method is described in the U.S. provisional application Ser. No. 60/760,382, filed Jan. 20, 2006, and entitled “Immunoconjugates with Improved Efficacy for the Treatment of Diseases,” the entire content of which is incorporated herein by reference.

Biologically-releasable bonds can also be used to construct the immunoconjugates of the present invention, such that the therapeutic agent(s) can be released from an antibody once it has entered the targeted cell. Numerous types of linking constructs are known, including simply direct disulfide bond formation between sulfhydryl groups contained on amino acids such as cysteine, or otherwise introduced into respective protein structures, and disulfide linkages using available or designed linker moieties.

A variety of disulfide-bond containing linkers are known, which can successfully be employed to conjugate therapeutic agents to antibodies. Certain linkers are preferred, such as, for example, sterically hindered disulfide bond linkers, due to their greater stability in vivo, thus preventing release of the toxin moiety prior to binding at the site of action. Another preferred cross-linking reagent is SMPT, although other linkers such as SATA, SPDP and 2-iminothiolane may also be employed.

Once conjugated, the immunoconjugates of the present invention can be purified to remove contaminants such as unconjugated therapeutic agents or antibodies. In many cases, it is important to remove unconjugated therapeutic agents because of the possibility of increased toxicity. Moreover, unconjugated antibodies may be removed to avoid the possibility of competition for the antigen between conjugated and unconjugated species. Numerous purification techniques can be used to provide conjugates to a sufficient degree of purity to render them clinically useful.

The present invention also features antibodies that are conjugated to a “receptor” (such as streptavidin) for utilization in cell pretargeting, where the antibody-receptor conjugates are administered to a subject of interest, followed by removal of unbound conjugate from the circulation using a clearing agent and then administration of a “ligand” (e.g., avidin) which is conjugated to a therapeutic agent (e.g., a radio nuclide).

In addition, the immunoconjugates of the present invention can be formulated as immunoliposomes. Liposomes containing antibodies can be prepared by methods known in the art, such as those described in Epstein et al., PROC. NATL. ACAD. SCI. USA, 82:3688 (1985); Hwang et al., PROC. NATL. ACAD. SCI. USA, 77:4030 (1980); and U.S. Pat. Nos. 4,485,045 and 4,544,545. Liposomes with enhanced circulation time are disclosed in U.S. Pat. No. 5,013,556. In one embodiment, an immunoliposome of the present invention is generated by the reverse phase evaporation method with a lipid composition comprising phosphatidylcholine, cholesterol and PEG-derivatized phosphatidylethanolamine (PEG-PE). Liposomes are extruded through filters of defined pore size to yield liposomes with the desired diameter. Antibody fragments (e.g., Fab′) can be conjugated to the liposomes as described in Martin et al., J. BIOL. CHEM., 257:286-288 (1982) via a disulfide interchange reaction. A therapeutic agent is optionally contained within the liposome.

In a preferred embodiment, the immunoconjugates of the present invention comprises an antibody which specifically recognizes a surface antigen of a pathogen, such as a virus, a bacterium, a yeast, a fungus, a parasite, or any other pathogenic or infectious microbe or organism. The present invention features the use of antibodies against the surface antigens of the following non-viral organisms: Streptococcus (e.g., Streptococcus agalactiae, Streptococcus pyogenes, Group C streptococci (beta hemolytic, occasionally alpha or gamma, e.g., Streptococcus anginosus or Streptococcus equismilis), Group D streptococci (alpha or gamma hemolytic, occasionally beta, e.g., Streptococcus bovis), Group E streptococci, Group F streptococci (beta hemolytic, e.g., Streptococcus anginosus), Group G streptococci (beta hemolytic, e.g., Streptococcus anginosus), Groups H and K through V streptococci, Viridans streptococci (e.g., Streptococcus mutans or Streptococcus sangui), Streptococcus faecalis, or Streptococcus pneumoniae); Staphylococcus (e.g., Staphylococcus epidermidis, Staphylococcus aureus, Staphylococcus saprophyticus, Staphylococcus haemolyticus, or Staphylococcus hominis); Actinobacillus (e.g., Actinobacillus lignieresi, Actinobacillus pleuropneumoniae); Actinomyces (e.g., Actinomyces bovis, Actinomyces israelii or Actinomyces naeslundii); Aerobacter (e.g., Aerobacter aerogenes); Alloiococcus (e.g., Alloiococcus otitidis); Anaplasma (e.g., Anaplasma marginale); Bacillus (e.g., Bacillus anthracis or Bacillus cereus); Bordetella (e.g., Bordetella pertussis or Bordetella parapertussis); Borrelia (e.g., Borrelia anserina, Borrelia recurrentis or Borrelia burgdorferi); Brucella (e.g., Brucella canis or Brucella melintensis); Campylobacter (e.g., Campylobacter jejuni); Chlamydia (e.g., Chlamydia psittaci, Chlamydia pneumoniae, Chlamydia trachomatis); Clostridium (e.g., Clostridium botulinum, Clostridium chauvoei, Clostridium difficile, Clostridium hemolyticium, Clostridium novyi, Clostridium perfringens, Clostridium septicum or Clostridium tetani); Corynebacterium (e.g., Corynebacterium equi, Corynebacterium diphtheriae, Corynebacterium pyogenes or Corynebacterium renale); Coxiella (e.g., Coxiella burneti); Cowdria (e.g., Cowdria ruminantium); Dermatophilus (e.g., Dermatophilus congolensis); Erysipelothrix (e.g., Erysipelothrix insidiosa or Erysipelothrix rhusopathiae); Escherichia (e.g., Escherichia coli); Francisella (e.g., Francisella tularensis); Fusiformis (e.g., Fusiformis necrophorus); Haemobartonella (e.g., Haemobartonella canis); Haemophilus (e.g., Haemophilus influenza, both typable and nontypable, or Haemophilus parainfluenzae); Helicobacter (e.g., Helicobacter pylori) Klebsiella (e.g., Klebsiella pneumoniae); Legionella (e.g., Legionella pneumophila); Leptospira (e.g., Leptospira interrogans); Listeria (e.g., Listeria monocytogenes); Moraxella (e.g., Moraxella bovis or Moraxella catarrhalis); Mycobacterium (e.g., Mycobacterium bovis, Mycobacterium leprae or Mycobacterium tuberculosis); Mycoplasma (e.g., Mycoplasma hyopneumoniae, Mycoplasma gallisepticum or Mycoplasma pneumoniae); Nanophyetus (e.g., Nanophyetus salmincola); Neisseria (e.g., Neisseria gonorrhoeae or Neisseria meninigitidis); Nocardia (e.g., Nocardia asteroides); Pasteurella (e.g., Pasteurella anatipestifer, Pasteurella haemolytica or Pasteurella multocida); Proteus (e.g., Proteus vulgaris or Proteus mirabilis) Pseudomonas (e.g., Pseudomonas aeruginosa); Rickettsia (e.g., Rickettsia mooseria, Rickettsia prowazekii, Rickettsia rickettsii or Rickettsia tsutsugamushi); Salmonella (e.g., Salmonella typhi or Salmonella typhimurium); Shigella (e.g., Shigella dysenteriae or Shigella boydii); Treponema (e.g., Treponema pallidum); Vibrio (e.g., Vibrio cholerae); or Yersinia (e.g., Yersinia enterocolitica or Yersinia pestis); protozoan species selected from Eimeria, Anaplasma, Giardia, Babesia, Trichomonas, Entamoeba, Balantidium, Plasmodium, Leishmania, Toxoplasma, Trypanosoma, Entamoeba, Trichomonas, Toxoplasmosa, or Pneumocystis; fungal species selected from Blastomyces, Microsporum, Aspergillis, Candida, Coccidiodes, Cryptococcus, Histoplasma or Trichophyton; and parasites such as trypanosomes, tapeworms, roundworms, and helminthes.

The present invention also features antibodies against the surface antigens of the following viruses: Paramyxoviridae (e.g., pneumovirus, morbillivirus, metapneumovirus, respirovirus or rubulavirus); Adenoviridae (e.g., adenovirus); Arenaviridae (e.g., arenavirus such as lymphocytic choriomeningitis virus); Arteriviridae (e.g., porcine respiratory and reproductive syndrome virus or equine arteritis virus); Bunyaviridae (e.g., phlebovirus or hantavirus); Caliciviridae (e.g., Norwalk virus); Coronaviridae (e.g., coronavirus or torovirus); Filoviridae (e.g., Ebola-like viruses); Flaviviridae (e.g., hepacivirus or flavivirus); Herpesviridae (e.g., simplexvirus, varicellovirus, cytomegalovirus, roseolovirus, or lymphocryptovirus); Orthomyxoviridae (e.g., influenza A virus, influenza B virus, influenza C virus, or thogotovirus); Parvoviridae (e.g., parvovirus); Picornaviridae (e.g., enterovirus or hepatovirus); Poxyiridae (e.g., orthopoxvirus, avipoxvirus, or Leporipoxvirus); Retroviridae (e.g., lentivirus or spumavirus); Reoviridae (e.g., rotavirus); Rhabdoviridae (e.g., lyssavirus, novirhabdovirus, or vesiculovirus); or Togaviridae (e.g., alphavirus or rubivirus). Preferred viral antigens include, but are not limited to, antigens from human immunodeficiency virus (HIV), human respiratory syncytial virus, influenza, herpes simplex virus 1 and 2, measles virus, hepatitis A virus, hepatitis B virus (HBV), hepatitis C virus (HCV), smallpox virus, polio virus, west Nile virus, coronavirus associated with severe acute respiratory syndrome, rotavirus, papilloma virus, papillomaviruses, Epstein-Barr virus (EBV), human T-cell lymphotropic virus type I, and Kaposi's sarcoma-associated herpesvirus.

Non-limiting examples of viral surface/envelope antigens include HIV surface/envelope antigens, HBV surface/envelope antigens, HCV surface/envelope antigens, EBV surface/envelope antigens, influenza surface/envelope antigens, and SARS associated coronavirus surface/envelope antigens. Specific examples include HIV gp120, HIV gp41, HBV surface protein antigen (e.g., the large (LHBs), middle (MHBs), or small (SHBs) surface protein), HCV E1, HCV E2, EBV gp350, EBV gp220, EBV gp85, EBV gp25, EBV gp42, influenza hemagglutinin, and influenza neuraminidase, coronavirus spike glycoprotein, and coronavirus hemagglutinin-acetylesterase.

Specific examples of preferred antibodies for the present invention include, but are not limited to, anti-HIV mAb 2F5, anti-HIV mAb 2G12, anti-HIV mAb 4E10, and anti-HIV mAb C108G. See Buchacher et al., AIDS RES HUM RETROVIRUSES, 10: 359-369 (1994); and Vijh-Warrier et al., J VIROL., 70: 4466-4473 (1996), both of which are incorporated herein by their entireties.

The present invention further features pharmaceutical compositions comprising the immunoconjugates of the present invention. A typical pharmaceutical composition of the present invention can be prepared by mixing an immunoconjugate of the present invention having the desired degree of purity with optional pharmaceutically acceptable carriers, excipients or stabilizers, in the form of lyophilized formulations or aqueous solutions. Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as TWEEN™, PLURONICS™ or polyethylene glycol (PEG). Supplementary active ingredients also can be incorporated into the pharmaceutical compositions of the present invention.

In many embodiments, a pharmaceutical composition of the invention includes a therapeutically effective amount of an immunoconjugate. As used herein, a “therapeutically effective amount” refers to an amount of an immunoconjugate effective to treat or prevent a viral infection in a mammal.

The immunoconjugate in a pharmaceutical composition of the present invention can be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Sustained-release preparations may be prepared. Suitable examples of sustained-release preparations include semipermeable matrices of solid hydrophobic polymers containing the immunoconjugate, which matrices are in the form of shaped articles, e.g. films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and γ-ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid.

A pharmaceutical composition of the present invention may be formulated in a neutral or salt form. Pharmaceutically-acceptable salts include the acid addition salts formed with the free amino groups of the protein or formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

Upon formulation, compositions or solutions will be administered in a manner compatible with the dosage formulation and in such amount as is therapeutically or prophylactically effective. The formulations are easily administered in a variety of dosage forms such as injectable solutions, drug release capsules and the like. For parenteral administration in an aqueous solution, for example, the solution can be suitably buffered if necessary and the liquid diluent first rendered isotonic with sufficient saline or glucose. These particular aqueous solutions are especially suitable for intravenous, intramuscular, subcutaneous, and intraperitoneal administration. In this connection, sterile aqueous media which can be employed will be known to those of skill in the art in light of the present disclosure. For example, one dosage could be dissolved in 1 ml of isotonic NaCl solution and either added to 1000 ml of hypodermoclysis fluid or injected at the proposed site of infusion, (see, for example, REMINGTON'S PHARMACEUTICAL SCIENCES (15th Edition), pages 1035-1038 and 1570-1580). Some variation in dosage will necessarily occur depending on the condition of the subject being treated.

The skilled artisan is directed to REMINGTON'S PHARMACEUTICAL SCIENCES (15th Edition), Chapter 33, in particular, pages 624-652, the entire contents of which are incorporated herein by reference. Some variation in dosage will necessarily occur depending on the condition of the subject being treated. The person responsible for administration will determine the appropriate dose for the individual subject.

Administration of a pharmaceutical composition of the present invention can be by way of any common route so long as the target tissue is available via that route. This includes oral, nasal, buccal, rectal, vaginal or topical. Alternatively, administration may be by orthotopic, intradermal, subcutaneous, intramuscular, intraperitoneal, intratumoral, circumferentially, catheterization, or intravenous injection.

A pharmaceutical composition can also be administered to a subject of interest (e.g., an HIV/HBV/HCV/EBV patient) parenterally or intraperitoneally. Solutions of the active compounds as free base or pharmacologically-acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations may contain a preservative to prevent the growth of microorganisms.

The pharmaceutical forms suitable for injectable use include sterile aqueous solutions or dispersions and sterile powders for the extemporaneous preparation of sterile injectable solutions or dispersions. In most cases, the form is sterile and fluid to the extent that easy syringability exists. It is preferably also stable under the conditions of manufacture and storage and preserved against the contaminating action of microorganisms, such as bacteria and fungi. The carrier can be a solvent or dispersion medium containing, for example, water, ethanol, polyol (for example, glycerol, propylene glycol, and liquid polyethylene glycol, and the like), suitable mixtures thereof, or vegetable oils. The proper fluidity can be maintained, for example, by the use of a coating, such as lecithin, by the maintenance of the required particle size in the case of dispersion, or by the use of surfactants. The prevention of the action of microorganisms can be brought about by various anti-bacterial or anti-fungal agents, for example, parabens, chlorobutanol, phenol, sorbic acid, thimerosal or the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions can be prepared by incorporating an immunoconjugate of the present invention in the required amount in an appropriate solvent with various other ingredients enumerated above, as required, followed by filtered sterilization. Generally, dispersions are prepared by incorporating the various sterilized active ingredients into a sterile vehicle, which contains the basic dispersion medium and the required other ingredients from those enumerated above. In the case of sterile powders for the preparation of sterile injectable solutions, the preferred methods of preparation are vacuum-drying or freeze-drying techniques which yield a powder of the active ingredient plus any additional desired ingredient from a previously sterile-filtered solution thereof.

For oral administration, an immunoconjugate of the present invention can be incorporated with excipients and used in the form of non-ingestible mouthwashes and dentifrices. A mouthwash may be prepared incorporating the active ingredient in the required amount in an appropriate solvent, such as a sodium borate solution (Dobell's Solution). Alternatively, an immunoconjugate of the present invention may be incorporated into an anti-septic wash containing sodium borate, glycerin and potassium bicarbonate. An immunoconjugate of the present invention may also be dispersed in dentifrices, including: gels, pastes, powders, or slurries. An immunoconjugate of the present invention may be added in a therapeutically or prophylactically effective amount to a paste dentifrice that may include water, binders, abrasives, flavoring agents, foaming agents, or humectants.

Upon administration, an immunoconjugate of the present invention binds to the targeted virus or pathogen. Entry of the virus/pathogen into the host cells also introduces the immunoconjugate into the cells, which subsequently destroys the cells (and, preferably, the bound virus) via the coupled therapeutic agent(s).

The immunoconjugates of the present invention can also be used to kill viruses or pathogens or their infected cells in vitro, through contacting or binding to the viruses or pathogens.

The foregoing description of the present invention provides illustration and description, but is not intended to be exhaustive or to limit the invention to the precise one disclosed. Modifications and variations are possible consistent with the above teachings or may be acquired from practice of the invention. Thus, it is noted that the scope of the invention is defined by the claims and their equivalents.

What is claimed is:

1. An immunoconjugate comprising an antibody coupled to a therapeutic agent, wherein said antibody specifically recognizes a surface or envelope antigen of a virus.

2. The immunoconjugate of claim 1, wherein said therapeutic agent is a radioactive isotope, a cytotoxic agent, or a prodrug.

3. The immunoconjugate of claim 1, wherein said therapeutic agent is selected from the group consisting of At²¹¹, I¹³¹, I¹²⁵, Y⁹⁰, Re¹⁸⁶, Re¹⁸⁸, Sm¹⁵³, Bi²¹², P³², and Lu¹⁷⁷.

4. The immunoconjugate of claim 1, wherein said therapeutic agent is selected from the group consisting of mitomycin C and pingyangmycin.

5. The immunoconjugate of claim 1, wherein said surface or envelope antigen is selected from the group consisting of:
an HIV surface or envelope antigen;
an HBV surface or envelope antigen;
an HCV surface or envelope antigen;
an EBV surface or envelope antigen;
an influenza surface or envelope antigen; and
a SARS associated coronavirus surface or envelope antigen.

6. The immunoconjugate of claim 1, wherein said surface or envelope antigen is selected from the group consisting of HIV gp120, HIV gp41, HBV surface protein antigen, HCV E1, HCV E2, EBV gp350, EBV gp220, EBV gp85, EBV gp25, EBV gp42, influenza hemagglutinin, and influenza neuraminidase, coronavirus spike glycoprotein, and coronavirus hemagglutinin-acetylesterase.

7. The immunoconjugate of claim 1, wherein said antibody is selected from the group consisting of anti-HIV mAb 2F5, anti-HIV mAb 2G12, anti-HIV mAb 4E10, anti-HIV mAb C108G, and anti-HBVsAg mAb.

8. The immunoconjugate of claim 16, wherein said antibody is selected from the group consisting of IgG, IgM, IgA, IgD, and IgE.

9. The immunoconjugate of claim 1, wherein said antibody is selected from the group consisting of full-length antibody, scFv, Fv, Fab, F(ab)₂, Fab′, diabody, triabody, and minibody.

10. The immunoconjugate of claim 1, wherein said antibody is a humanized or human antibody.

11. The immunoconjugate of claim 1, wherein said antibody is a rat, murine, cow, dog, sheep, goat, guinea pig, rabbit, macaque, chimpanzee, or chimeric antibody.

12. A pharmaceutical composition comprising the immunoconjugate of claim 1.

13. A method for treating or preventing infection of a virus, comprising contacting said virus with the immunoconjugate of claim 1, wherein said immunoconjugate comprises an antibody specifically recognizing a surface or envelope antigen of said virus.

14. A method for treating or preventing infection of a virus, comprising administering the immunoconjugate of claim 1 to a subject of interest, wherein said immunoconjugate comprises an antibody specifically recognizing a surface or envelope antigen of said virus.

15. The method of claim 14, wherein said subject of interest is a human or an animal.
